(19) United States
(12) Patent Application Publication
(10) Pub. No.: US 2014/0199739 A1
(43) Pub. Date: Jul. 17, 2014

(54) NOVEL STRAINS OF MICROALGAE OF THE ISOCHRYSIS GENUS FOR PRODUCING EPA AND DHA IN A MIXOTROPHIC MODE

(71) Applicants: Pierre Calleja, Bordeaux (FR); Khadidja Romari, Clermont-Ferrand (FR)

(72) Inventors: Pierre Calleja, Bordeaux (FR); Khadidja Romari, Clermont-Ferrand (FR)

(73) Assignee: FERMENTALG, Libourne (FR)

(21) Appl. No.: 14/127,389

(22) PCT Filed: Jun. 20, 2012
(86) PCT No.: PCT/FR2012/051386

§ 371 Date: Feb. 20, 2014

(30) Foreign Application Priority Data
Jun. 21, 2011 (FR) 1155443

(51) Int. Cl. C12P 7/64

(52) U.S. Cl. CPC C12P 7/6427
USPC 435/134; 435/257.1

(57) ABSTRACT

Novel strains of microalgae belonging to the Isochrysis genus, allowing optimum production of polyunsaturated fatty acids, notably EPA, in mixotrophic mode, and a method for selecting and culturing such strains, using a discontinuous supply of light in the form of flashes are described.

The invention relates to a method for the culture and selection of microalgae strains belonging to the Isochrysis genus involving a discontinuous supply of light in the form of flashes, and to selected strains of Isochrysis that are particularly suitable for the production of polyunsaturated fatty acids and, notably, EPA (eicosapentaenoic acid) in mixotrophic culture mode.

### PREAMBLE

It is known that microalgae are photosynthetic microorganisms having an autotrophic character, i.e. they have the capacity to grow autonomously by photosynthesis.

Microalgae develop both in marine aquatic media and in fresh or brackish waters, as well as in various land habitats.

Most of the microalgae species found in freshwater or the oceans are strictly autotrophic, i.e. they can only grow by photosynthesis. For these species, the presence in their environment of carbon-containing substrates or organic matter is not favorable to them and even tends to inhibit their growth.

However, a certain number of microalgae species of very varied families and origins are found to be not strictly autotrophic. Thus, some of them, said to be heterotrophic, are capable of developing in the total absence of light, by fermentation, i.e. by using organic matter.

Other microalgae species, for which photosynthesis remains essential for their development, are capable of benefiting both from photosynthesis and from the organic matter present in their environment. These intermediate species, said to be mixotrophic, can be cultured in the presence of both light and organic matter.

This particularity of so-called [symbol] mixotrophic [symbol] algae seems to be related to their metabolism, which allows them to carry out photosynthesis and fermentation simultaneously. Both types of metabolism co-exist with a positive overall effect on the growth of the algae [Yang C. et al. (2000) Biochemical Engineering Journal 6: 87-102].

At present, the classification of algae is still widely based on morphological criteria and on the type of photosynthetic pigments which their cells contain. Consequently, it is not very indicative of the autotrophic, heterotrophic or mixotrophic character of algae species, whereas the latter cover a very great diversity of species and forms [Dubinsky et al. 2010, Hydrobiologia, 639:153-171].

Microalgae are currently the subject of numerous industrial projects since some species are capable of accumulating or secreting major quantities of lipids, in particular polyunsaturated fatty acids.

Among these polyunsaturated fatty acids, certain highly unsaturated acids from the series of Omega-3s (HUFAs or PUFA-ω3), in particular eicosapentaenoic acid (EPA, C20:5 ω3) and docosahexaenoic acid (DHA, C22:6 ω3) have a recognized nutritional importance and have strong potential in terms of therapeutic applications [Horrocks L. A. et al. (2000) Health Benefits of DHA. Pharmacol. Res. 40: 211-225].

Fish oils from the fish industry are currently the main commercial source of these types of fatty acids. However, while these oils find new applications (food supplement in aquaculture, incorporation into margarines), marine halieutic resources are becoming scarce because of intensive fishing activity.

New sources of EPA and DHA, therefore, have to be sought in order to meet, in the future, the increasing demand for these types of polyunsaturated fatty acids.

In addition to their capability of synthesizing fatty acids de novo, microalgae provide several advantages compared with fish oils: they may be cultured in vitro under controlled conditions, which allows production of a biomass of a relatively constant biochemical composition, and, in addition, unlike fish oils, they do not have an unpleasant smell and their lipids contain little or no cholesterol.

Finally, the lipids produced by microalgae have a simpler fatty acid profile than that of fish oils, which limits the steps for separating the fatty acids of interest.

The taxonomic classification of eukaryotic algae contains 14 phyla. Large variations exist among the different species of the different classes making up these phyla as regards the polyunsaturated fatty acid content of the microalgae. Moreover, the relative proportions of EPA and DHA in the lipid profiles vary according to the species and the culture conditions [Yongmanitchai, W. and Ward, O. P. (1989) Omega-3 fatty acids: alternative sources of production. Process. Biochem. 24 :117-125].

In the perspective of an industrial utilization of microalgae, it is the species of heterotrophic or mixotrophic character which currently arouse most interest by industrialists. The reduced dependency of this type of microalgae on light makes it possible to envisage their culture in closed, large tanks, as is carried out in fermenters for bacteria or yeasts.

Compared with conventional cultures in autotrophic mode, these new culture modes make possible savings in space and energy related to the supply of a weaker light intensity and less intensive mixing of the cultures.

Nevertheless, numerous species of microalgae grown conventionally in autotrophic mode prove to be unable to be cultured in heterotrophic mode.

This is, in particular, the case of microalgae of the Isochrysis genus, which are flagellate marine microalgae of brown colour belonging to the class of Prymnesiophyceae.

The microalgae of the Isochrysis genus are widely used in fish farming in hatcheries of fish, shrimps, shellfish and molluscs as DHA-rich food supplement. These microalgae are generally marketed in the form of long-life concentrates of microalgae preparations (Algues instantanées®, 871 East Hamilton Ave, Campbell, Calif. 95008, USA). The particular aspect of these microalgae is that they accumulate their fatty acids in an intra-cellular mode in the form of lipid inclusions. Since their walls are relatively thin, several studies place Isochrysis among those microalgae best lending themselves to the extraction of their lipids on an industrial scale.

The strains of Isochrysis currently described are light-dependent, which explains why they cannot be cultured in heterotrophic mode. However, some studies (Liu, C-P. and Lin, L-P. (2001): Ultrastructural study and lipid formation of Isochrysis sp. (2001) Bot. Bull. Acad. Sin. 42: 207-214] have determined that some strains, in particular, the marketed strain CCMP1324, can be cultured in mixotrophic mode in artificial seawater (3.2% NaCl) at 25° C., pH=8, with a continuous supply of light of 10 klux (more than 160 μmol.m⁻².s⁻² expressed in photons) in the presence of a concentration of 10 to 50 mM sodium acetate (carbon-containing substrate). Under such conditions, a total biomass of 4 g/l by dry weight of microalgae was obtained, with optimal DHA production corresponding to 16 mg per litre of culture.

This being the case, to the applicant's knowledge, no strain of Isochrysis sp. has shown itself to be capable of producing EPA under these conditions.

It is thus, unexpectedly, after many experiments performed with varied strains under different mixotrophic conditions, that the applicant has managed to select and culture strains of Isochrysis capable of producing EPA in mixotrophic mode.

This method, subject of the present invention, more particularly relates to the culture of microalgae under mixotrophic conditions in the presence of discontinuous illumination, notably in the form of flashes.

The close alternation of illuminated phases and dark phases, generally perceived as stressful for microalgae, surprisingly made it possible to select Isochrysis strains capable of producing both EPA and DHA in mixotrophic mode.

With this method, it was therefore possible, for the first time, to produce EPA from strains of Isochrysis under mixotrophic conditions.

One strain (FCC 1111) representing novel strains of Isochrysis, selected and cultured according to the invention, was deposited at the CCAP (Culture Collection of Algae and Protozoa, Scottish Association for Marine Science, Dunstaffnage Marine Laboratory, Oban, Argyll PA371QA, Scotland, United Kingdom) according to the provisions of the Treaty of Budapest, on May 27, 2011 under the accession number CCAP 927/16.

The use of the strains and culture method of the invention therefore opens up the perspective prospect of industrial production of polyunsaturated fatty acids, in particular, EPA and DHA, using strains of the Isochrysis genus, in fermenters benefiting from reduced light and energy savings.

The different aspects and advantages of the invention are detailed below.

### DETAILED DESCRIPTION

The present invention firstly concerns novel strains of microalgae of the genus Isochrysis (Isochrysis sp.) characterized in that they are capable of producing EPA under mixotrophic culture conditions.

To the applicant's knowledge, the strains of Isochrysis isolated according to the invention are the first described as being capable, under mixotrophic conditions, to produce significant quantities of EPA, which may represent more than 5%, even more than 10% of the total lipids contained in the microalgae.

These novel strains of Isochrysis were isolated and selected in accordance with the selection and culture methods detailed below.

One strain representing strains of Isochrysis according to the invention is the FCC 1111 strain, deposited at the CCAP on May 27, 2011 under the accession number CCAP 927/16. This strain is characterized in that it is capable of producing EPA in mixotrophic culture mode.

According to ongoing taxonomic analyses, this strain belongs to the Isochrysis genus [Parke, M. 1949 (1949): Studies on marine flagellates. Journal of the Marine Biological Association of the United Kingdom 28: 255-288]. However, taking into account that the main species Isochrysis galbana, Isochrysis litoralis or Isochrysis maritima are phylogenetically close, it has not yet been possible to determine definitively the exact species to which strain FCC 1111 belongs. On this account, the invention concerns any species of Isochrysis capable of producing EPA in mixotrophic culture mode, such as described in this application.

The culture in mixotrophic mode of Isochrysis according to the invention is preferably conducted in a culture environment of f/2 type [Guillard, R. R. and Ryther, J. H. (1962): Studies on marine planktonic diatoms. I. Cyclotella nana Hustedt and Detonula confervacaea (Cleve) Gran. Canadian Journal of Microbiology 8: 229-239], in the presence of at least 5 mM, preferably, at least 10 mM, more preferentially, at least 20 mM, and still more preferentially, more than 50 mM of a carbon-containing substrate. This carbon-containing substrate, in pure form or in a mixture, preferably contains glucose, cellulose (or cellulose derivatives), starch, lactose, sucrose, acetate and/or glycerol.

More specifically, the culture in mixotrophic mode of this microalga is preferentially conducted in the presence of 10-200 mM and more preferentially between 20 and 50 mM of carbon-containing substrate. Preferably, the carbon-containing substrate present in the culture environment contains at least 5 mM glycerol or lactose.

A continual substrate supply is ensured during the culture, in order to allow the cells to accumulate a significant concentration of lipids. Additional substrate is added to the culture medium during the culture method so as to maintain a constant concentration. Culture may thus be carried out in the presence of cumulated concentrations of carbon-containing substrate from 5 mM to 1 M, preferably, from 50 mM to 800 mM, more preferentially, from 70 mM to 600 mM, and still more preferentially, from 100 mM to 500 mM.

This carbon-containing substrate may consist of mixtures of complex molecules or a mixture of substrates. The products from the biotransformation of starch, for example, from corn, wheat or potato, notably, starch hydrolyzates, which consist of small sized molecules, may be carbon-containing substrates suitable for culturing microalgae in mixotrophic mode according to the invention.

The invention further concerns a culture method for microalgae of the Isochrysis genus in mixotrophic mode with a view to producing polyunsaturated fatty acids, notably, EPA. The effect of this method is to enrich the microalgae of the Isochrysis genus in polyunsaturated fatty acids, which generally translates as an increase in the proportion of EPA or DHA contained in the total lipids produced by said microalgae.

Surprisingly, the yield of the microalgae in EPA is higher when the microalgae are cultured in the presence of a variable or discontinuous supply of light, in other words, when the light flux applied to the microalgae culture is variable or discontinuous over time.

Contrary to common beliefs, it appeared that that variable or discontinuous illumination of the cultures, in particular, when used in culture in mixotrophic mode, had a favorable impact on the development of algae and made it possible to increase their productivity, notably as far as their lipid production is concerned.

Without being bound by theory, the inventor believes that a discontinuous or variable light supply to the microalgae has the effect of causing a [symbol] stress [symbol] favorable to the synthesis of lipids. This phenomenon may partly be explained by the fact that, in nature, microalgae tend to accumulate lipid reserves to withstand the constraints of their environment.

By discontinuous illumination, it is meant illumination punctuated with periods of darkness. The periods of darkness may be more than one quarter of the time, preferably, half or more of the time, during which the algae are cultured.

According to a preferred aspect of the invention, the illumination is discontinuous and, more preferentially, in the form of flashes, i.e. over periods of short durations. The successive illumination phases are then generally comprised between 5 seconds and 10 minutes, preferably, between 10 seconds and 2 minutes, and more preferentially, between 20 seconds and 1 minute.

According to another embodiment of the invention, the illumination may be variable, which means that the illumination is not interrupted by phases of darkness, and the light intensity varies over time. This light variation may be periodical, cyclic, even random.

According to the invention, the illumination may vary continuously, i.e. the light intensity is not constant and permanently varies over time (dμmol(photons)/dt≠0).

According to the invention, it is also possible to have a light supply combining continuous and discontinuous illumination phases.

The invention particularly concerns a culture method for microalgae of the Isochrysis genus, characterized in that said algae are grown in the dark with a discontinuous or variable light supply over time, the intensity of which, in micromoles of photons, varies by an amplitude of 10 μmol. m⁻².s⁻² or higher several times per hour, preferably, 50 μmol.m⁻².s⁻² or higher, more preferentially, 100 μmol.m⁻².s⁻¹ or higher. The common point of these different discontinuous or variable illumination modes lies in the fact that, according to the invention, the intensity of the light brought to the algae in culture, expressed in micromoles of photons per second per square metre (μmol.m⁻².s⁻¹), varies at least once within the same hour. The amplitude of this variation of light intensity is generally greater than 10 μmol.m⁻².s⁻², preferably, 20 μmol.m⁻².s⁻¹ or higher, more preferentially, 50 μmol.m⁻².s⁻¹ or higher. In other words, every hour and preferably several times within the hour, the light intensity attains a high value and a low value, the difference between these values being equal to or greater than that indicated above. Preferably, said light intensity successively attains the values 50 μmol.m⁻².s⁻² and 100 μmol.m⁻².s⁻² every hour, more preferentially, the values 0 and 50 μmol.m⁻².s⁻², and further preferentially, the values 0 and 100 μmol.m⁻².s⁻².

It is known that 1 μmol.m⁻².s⁻² corresponds to 1 μE m⁻².s⁻¹ (Einstein), a unit often used in the literature.

Preferably, and according to the knowledge of one skilled in the art, the intensity of the light applied to the culture may be increased as a function of cell density. For example, at the start of culture, the flashes may last seconds, for example, at a light intensity of 20-50 μmol.m⁻².s⁻², later, when culture becomes more dense, the length of the flashes may be increased to 20 seconds, at an intensity of 50-100 μmol.m⁻².s⁻². In the final culture phase, the flashes may have a length of 30 seconds and an intensity of 100-200 μmol. m⁻².s⁻².

The light supply to the cultures may be obtained by lamps distributed around the external wall of the fermenters. A clock triggers these lamps for defined illumination times. The fermenters are preferentially located in a temperature-controlled enclosure, shielded from daylight, whose ambient temperature may be controlled.

As the applicant could ascertain, the fact that the thus selected strains have good growth capabilities in mixotrophic mode, in the presence of a discontinuous light, predisposes said strains to higher production of polyunsaturated fatty acids, notably EPA.

The culture method according to the invention thus allows selection of strains of Isochrysis of mixotrophic character, having a high yield of polyunsaturated fatty acids and capable of producing EPA in mixotrophic mode, such as the strain FCC 1111, deposited at the CCAP under accession number CCAP 927/16.

This method generally comprises one or more of the following steps:
culturing various strains of the Isochrysis genus in darkness with a discontinuous or variable supply of light over time, the intensity of which in micromoles of photons, preferably varies by an amplitude equal to or higher than 50 μmol.m⁻².s⁻¹ at a rate of at least once per hour;
maintaining said culture over several generations;
isolating the strain(s) for which the number of cells has most increased during said generations.

To screen the strains, various Isochrysis strains may be cultured, in parallel, on microplates, in the same enclosure, with a precise monitoring of the conditions and of the development of the various cultures. It is, thus, easy to determine the response of the various strains to discontinuous illumination and, if necessary, upon adding one or several carbon-containing substrates into the culture medium. The strains, which react favorably to the discontinuous illumination and to the carbon-containing substrates, generally provide a better yield for the production of lipids in terms of quality (polyunsaturated fatty acids more abundant in the lipid profile) and in terms of quantity (the lipids contain a higher proportion of EPA).

The microalgae may be selected in a fermenter from a diversified pool of microalgae, and from which one aims to select the variants advantaged by the selection mode according to the invention, combining discontinuous or variable light with mixotrophic culture conditions. In this case, the culture is carried out by maintaining the microalgae in cultures over many generations, and then isolation of the components which have become a majority in the culture medium, is performed at the end of the culture.

The culture method according to the invention is more particularly characterized in that culture of the strains is carried out over several generations, preferably in mixotrophic mode, and, in that the cells loaded with lipids are harvested.

The invention thus also relates to the production of the lipids, notably fatty acids, via the culture of microalgae of the Isochrysis genus with a mixotrophic character, preferably cultured or selected according to the methods mentioned earlier, and then, the recovery of the thus cultured microalgae to extract therefrom the lipid content, in particular, EPA.

The methods for selectively extracting EPA and DHA are known to one skilled in the art and are, for example, described by Bligh, E. G. and Dyer, W. J. [A rapid method of total lipid extraction and purification (1959) Can. J. Biochem. Physiol 37:911-917].

The invention also relates to microalgae of the Isochrysis genus enriched in polyunsaturated fatty acids, which may be obtained according to the method of the invention, as described earlier. The total lipids of such microalgae generally comprise more than 20%, often more than 40% and sometimes even more than 50% EPA. Said microalgae may be used as a food supplement, in particular for fish farming.

### EXAMPLE

### Culture of Isochrysis Strains in a Bioreactor:

The cultures are grown in 2 L usable capacity fermenters (bioreactors) with dedicated automatic equipment with computerized supervision. The pH of the system is adjusted by adding base (1N sodium hydroxide) and/or acid (1N solution of sulphuric acid). The culture temperature is set to 22° C. Stirring is achieved using 3 stirring rotors placed on the shaft according to the Rushton configuration (three-blade propellers with down pumping). The stirring rate and the aeration flow rate are regulated to a minimum of 100 rpm and a maximum of 250 rpm and Qmini=0.5 vvm/Qmaxi=2 vvm respectively. The bioreactor is equipped with an external lighting system surrounding the transparent tank. The light intensity and the light cycles are controlled by dedicated automatic equipment with computerized supervision.

The reactors are inoculated with a preculture prepared on a mixing plate (140 rpm) in a controlled-temperature enclosure (22° C.) and illuminated continuously at 100 μE m⁻².s⁻¹. Precultures and cultures in bioreactors are prepared in f/2 medium supplemented with 10 μg/L Biotin and vitamin B12. The organic carbon used for the mixotrophic culture in a bioreactor is glycerol at final concentrations of between 20 and 30 g/L. The carbon-containing organic substrate is added to the culture medium in “fed-batch” mode.

### Monitoring of Cultures

The total biomass concentration is monitored by measuring the dry mass (filtration on a Whatman GFC filter, and then oven drying in vacuo at 65° C. and −0.8 bars, for a minimum of 24 h before weighing).

Regarding the quantification of the total number of lipids, 10⁷ cells/mL were extracted. Methods for extracting lipids are known to one skilled in the art and are, for example, described by Bligh, E. G. and Dyer, W. J. [A rapid method of total lipid extraction and purification (1959) Can. J. Biochem. Physiol 37:911-917].

### Flashing Light

The light supply in the bioreactor cultures was obtained by LED lamps distributed around the external wall of the fermenters. A clock triggers these LEDs for illumination times or flashes of between 20 and 200 μE m⁻².s⁻¹. Throughout the culture period, the length of the illumination times is between 15 and 30 seconds. The intensity and length of illumination time vary in relation to cell density. The total culture time is about 10 days. For the first three culture days, the flashes last 15 seconds at an intensity of 30 μE m⁻².s⁻¹. From the fourth day, the duration of the flashes is 20 seconds at an intensity of 75 μE m⁻².s⁻¹. For the last three culture days, the flashes last 30 seconds at an intensity of 150 μE m⁻².s⁻¹.

The light intensity of the flash system used in mixotrophy is the same as that used in autotrophy (control).

| Strain Isochrysis sp. | Mixotrophy with flashes |
|---|---|
| Biomass (% relative to autotrophy) | +30% |
| Total lipids (% relative to autotrophy) | +20% |
| EPA (% relative to autotrophy) | +20% |
| DHA (% relative to autotrophy) | +5% |

1. Method for producing EPA (eicosapentaenoic acid), characterized in that it comprises the culture in mixotrophic mode of a microalga of the Isochrysis genus, and the recovery of the biomass thus formed.

2. Method according to claim 1, characterized in that the culture in mixotrophic mode of said microalga of the Isochrysis genus is carried out in the presence of a carbon-containing substrate containing at least 5 mM, preferably, at least 10 mM, and more preferentially, at least 20 mM glucose, cellulose, starch, lactose, sucrose, acetate and/or glycerol.

3. Method according to claim 2, characterized in that said carbon-containing substrate contained in the culture medium comprises at least 5 mM glycerol.

4. Method according to claim 2, characterized in that said carbon-containing substrate contained in the culture medium comprises at least 5 mM lactose.

5. Method according to claim 1, characterized in that it comprises a step for extracting polyunsaturated fatty acids, which have accumulated in the microalgae during their growth.

6. Method according to claim 1, characterized in that it comprises the following steps:
culturing one or several strains of the Isochrysis genus in darkness with a discontinuous or variable supply of light over time, the intensity of which in micromoles of photons varies by an amplitude of more than 50 μmol.m⁻².s⁻¹, at a rate of at least once per hour;
maintaining said culture over several generations in the presence of a carbon-containing substrate in the culture environment;
harvesting the thus obtained Isochrysis cells.

7. Method according to claim 6, characterized in that the supply of light is in the form of flashes.

8. Method according to claim 7, characterized in that the flashing consists of successive illumination phases with a duration comprised between 5 seconds and 10 minutes, preferably, between 10 seconds and 2 minutes, more preferentially, between 20 seconds and 1 minute.

9. Method according to claim 1, characterized in that said microalga of the Isochrysis genus, is capable of producing EPA in mixotrophic mode, such as the FC 1111 strain, deposited on May 27, 2011 at the CCAP (Culture Collection of Algae and Protozoa) under accession number CCAP 927/16.

10. Method for selecting isolated strains of Isochrysis genus capable of producing EPA in mixotrophic mode, characterized in that it comprises a step for producing EPA by said strains according to claim 1.

11. Microalga of the Isochrysis genus enriched in EPA, which may be obtained in mixotrophic culture mode following the method according to claim 1.

12. Microalga of the Isochrysis genus cultured in mixotrophic mode, characterized in that the total lipids thereof contain more than 5%, preferably, more than 10%, more preferentially, more than 20% EPA.

13. Microalga of the Isochrysis genus according to claim 11, characterized in that the total lipids thereof also comprise at least 5% DHA.

14. Microalga characterized in that it consists of an isolated strain of the Isochrysis genus corresponding to strain FC 1111, deposited on May 27, 2011 at the CCAP (Culture Collection of Algae and Protozoa) under accession number CCAP 927/16.

15. Microalga of the Isochrysis genus according to claim 12 characterized in that the total lipids thereof also comprise at least 5% DHA.
